(19) United States
(12) Patent Application Publication
(10) Pub. No.: US 2010/0305368 A1
(43) Pub. Date: Dec. 2, 2010

(54) Combined Formose/Transfer Hydrogenation Process for Ethylene Glycol Synthesis

(76) Inventors: Robert H. Grubbs, South Pasadena, CA (US); Patricio E. Romero, Pasadena, CA (US)

Correspondence Address: GREENLEE SULLIVAN P.C., 4875 PEARL EAST CIRCLE, SUITE 200, BOULDER, CO, 80301, US

(21) Appl. No.: 12/680,316

(22) PCT Filed: Oct. 10, 2008
(86) PCT No.: PCT/US08/79450

§ 371 Date: Aug. 11, 2010

Related U.S. Application Data
(60) Provisional application No. 60/998,505, filed on Oct. 11, 2007.

(51) Int. Cl. C07C 29/14

(52) U.S. Cl. 568/881

(57) ABSTRACT

The present invention provides a process for the production of a glycol via tandem self condensation of formaldehyde via formoin condensation and transfer hydrogenation of the reaction products of the formoin condensation. In some aspects, synthetic processes of the present invention utilize a combination of a N-heterocyclic carbene catalyst and a transition metal hydrogen-transfer catalyst providing enhanced selectivity and increased yields for the production of ethylene glycol relative to conventional synthetic approaches based on formoin condensation.

### CROSS-REFERENCE TO RELATED APPLICATIONS

This application claims the benefit of U.S. Provisional Patent Application No. 60/998,505, filed Oct. 11, 2007, which is hereby incorporated by reference to the extent not inconsistent with the present disclosure.

### STATEMENT REGARDING FEDERALLY SPONSORED RESEARCH OR DEVELOPMENT

Not applicable.

### BACKGROUND OF INVENTION

Ethylene glycol is an important industrial chemical, reagent and commercial product. Ethylene glycol has been used as a starting material for the production of edible carbohydrates and in the prebiotic synthesis of carbohydrates. It has been used in the laboratory for many applications including precipitation of proteins and to protect functional groups during organic synthesis. Additionally, ethylene glycol is a major constituent of coolants, antifreeze and deicers due to its low freezing point.

Due to its chemical and commercial importance, synthetic pathways for the production of ethylene glycol and carbohydrates from simple starting materials have been sought for some time. U.S. Pat. No. 4,170,605 to Williamson et al. discloses the production of ethylene glycol from carbon monoxide and hydrogen using a ruthenium complex catalyst and pyridine base ligand. U.S. Pat. No. 2,451,333 to Gresham et al. discloses the use of ruthenium-based catalysts for the production of ethylene glycol from hydrogen, carbon monoxide and formaldehyde. The production of sugars and other carbohydrates from formaldehyde, known as the formose reaction, has been known since the 19^{th} century. The first step in the formose reaction, known as the formoin condensation, is the self-condensation of formaldehyde to form reaction products including glycolaldehyde. The formoin condensation has historically been catalyzed by calcium hydroxide, cyanide and imidazolium or thiazolium salts.

While early attempts at production of ethylene glycol from simple starting materials were able to identify synthetic pathways, these synthetic routes are not currently commercially viable due to low selectivity and reproducibility as well as the availability of alternative pathways involving more complex starting materials. Niitsu and co-workers [T. Niitsu, M. M. Ito, and H. Inoue, “Analysis of the Formose Reaction System,” Journal of Chemical Engineering of Japan, vol. 24, pgs. 480-485, (1992)] have shown that the formose reaction can be unselective and irreproducible and may yield a variety of hydrocarbons with carbon backbones ranging from C₂ to C₇. In addition, the selectivity and reproducibility of the formose reaction employing early generation catalysts may be sensitive to small changes in experimental conditions such as temperature and pressure. This sensitivity has made predicting the major products of the formose reaction employing early generation catalysts very difficult and has hindered the commercial viability of this reaction.

Recent progress in catalyst development has resulted in the potential to provide a cost effective and commercially practicable synthetic route for ethylene glycol production. Two types of catalysts have been independently developed which may catalyze different steps in the formation of ethylene glycol from formaldehyde. The first type of catalysts are nitrogen-containing heterogeneous rings which have been found to catalyze the self-condensation of formaldehyde to form glycolaldehyde and other products with high selectivity. [J. M. D. Storey and C. Williamson, “Imidazole based solid-supported catalysts for the benzoin condensation,” Tetrahedron Letters, vol. 46, pgs. 7337-7339 (2005); D. Enders and T. Balensiefer, “Nucleophilic Carbenes in Asymmetric Organocatalysis,” Accounts of Chemical Research, vol. 37, pgs. 534-541 (2004); T. Matsumoto and S. Inoue, “Selective Formation of Triose from Formaldehyde catalysed by Ethylbenzothiazolium Bromide,” Journal of the Chemical Society, Chemical Communications, pgs. 171-172 (1983)]. The second type of catalysts are ruthenium-based carbene catalysts which have been shown to catalyze the transfer hydrogenation of carbonyl groups to alcohol groups. [O. Pamies and J-E. Backvall, “Combination of Enzymes and Metal Catalysts. A Powerful Approach in Asymmetric Catalysis,” Chemical Reviews, vol. 103, pgs. 3247-3261, (2006)].

U.S. Pat. No. 5,118,883 (Gehrer et al.) discloses a process for the two-step preparation of glycols from formaldehyde. The first step discloses involves the condensation of formaldehyde (the formose reaction) and the second step disclosed involves the catalytic hydrogenolysis of the formose reaction product. The reference further discloses that the formose reaction may be catalyzed in general by basic catalysts such as CaO, SrO, BaO, TlOH, or PbO, organic bases, or thiazolium or imidazolium salts. The second disclosed hydrogenolysis step may be catalyzed by catalyst used for carbonyl compounds in the presence of hydrogen gas at a pressure of 1 to 1000 bar. The reference discloses, however, that the hydrogenolysis reaction is preferably carried out using a ruthenium or nickel catalyst.

U.S. Pat. No. 5,386,062 (Teles et al.) discloses a process for the catalytic preparation of condensation products of formaldehyde. The reference discloses use of a carbene catalyst produced in the presence of an auxiliary base from a triazolium salt which may include optionally substituted aryl groups. The reference further discloses catalysis of the condensation of formaldehyde to form glycolaldehyde, glyceraldehyde, and tetroses.

O. Pamies and J-E. Backvall, “Combination of Enzymes and Metal Catalysts. A Powerful Approach in Asymmetric Catalysis,” Chemical Reviews, vol. 103, pgs. 3247-3261, (2006) (Pamies et al.) disclose different pathways for transfer hydrogenation reactions catalyzed by ruthenium based catalysts. The reference describes the racemization of an alcohol catalyzed by a ruthenium compound. The reference discloses that a ketone may be reduced by an intermediate of the ruthenium catalyst to form a racemic alcohol in the last step of the racemization.

U.S. Pat. No. 4,321,414 (Costa) discloses a process by which glycolaldehyde is hydrogenated to form ethylene glycol by reacting glycolaldehyde and hydrogen gas at a pressure of 15-2500 psig in the presence of a ruthenium catalyst disclosed in formula I of the reference. The ruthenium catalysts disclosed by the reference contain many substituent ligands species such as phosphorous or arsenic based substituent ligands, halide or pseudohalide substituent ligands and carbonyl based substituent ligands. Example 2 of the reference demonstrates reasonable yields and selectivity of the disclosed ruthenium compound catalysis of glycolaldehyde to form ethylene glycol.

As will be understood from the foregoing, there is currently a need in the art for a synthetic platform for the catalytic conversion of formaldehyde to ethylene glycol which is selective, reproducible and commercially practicable.

### SUMMARY OF THE INVENTION

The present invention provides a process for the production of a glycol via tandem self condensation of formaldehyde via formoin condensation and transfer hydrogenation of the reaction products of the formoin condensation. In some aspects, synthetic processes of the present invention utilize a combination of a N-heterocyclic carbene catalyst and a transition metal hydrogen-transfer catalyst providing enhanced selectivity and increased yields for the production of ethylene glycol relative to conventional synthetic approaches based on formoin condensation. For example, processes of the present invention using Enders carbene (and/or Enders carbene as its methanol adduct) and Shvo's catalyst for the catalysis of formoin condensation and transfer hydrogenation processes, respectively, provide high yields of ethylene glycol (e.g., as great as 18 to 20%) and a reduction of unwanted reaction byproducts, such as glycolaldehyde dimethylacetal. The synthetic processes of the present invention also enable the one pot synthesis of glycol from formaldehyde via coupled catalytic formoin condensation and transfer hydrogenation process, particularly ethylene glycol, thereby eliminating the need for time and resource intensive separation and purification processes for reaction intermediates in the synthetic protocol.

In an embodiment, the invention provides a process for the production of a glycol from formaldehyde comprising the steps of: (i) contacting formaldehyde with a N-heterocyclic carbene catalyst thereby generating one or more reaction products; and (ii) providing a transition metal hydrogen-transfer catalyst in contact with at least a portion of the one or more reaction products, thereby producing glycol. In an embodiment of this aspect, the invention provides a process for the production of ethylene glycol from formaldehyde. In an embodiment, for example, the step of contacting formaldehyde with the N-heterocyclic carbene catalyst initiates catalytic condensation of formaldehyde to generate a glycolaldehyde reaction product, and the step of providing the transition metal hydrogen-transfer catalyst in contact with at least a portion of the one or more reaction products initiates catalytic transfer hydrogenation of glycolaldehyde to generate ethylene glycol as a primary reaction product. In an embodiment, the process steps of the present invention are carried out sequentially in a single reactor in one or more solvents, optionally in the presence of one or more additives provided to enhance glycol formation. In an embodiment, the step(s) of contacting formaldehyde with a N-heterocyclic carbene catalyst; and/or providing a transition metal hydrogen-transfer catalyst in contact with at least a portion of the one or more reaction products is carried out in the presence of a base.

In some embodiments, the transition metal hydrogen-transfer catalyst is provided in contact with the one or more reaction products in the presence of a solvent or other additive that facilitates the transfer hydrogenation of reaction products generated in the formoin condensation step. In some embodiments, for example, the present processes include the step of contacting the transition metal hydrogen-transfer catalyst and one or more of the reaction products in the presence of an alcohol, such as methanol, so as to achieve efficient transfer hydrogenation of a glycolaldehyde reaction product, thereby resulting in production of ethylene glycol. The molar ratio of formaldehyde provided to the reactor to alcohol provided to the reactor, such as methanol, is optionally in the range 0.01 to 0.70, and preferentially for some applications 0.08 to 0.16. Other solvents and/or additives useful in the present processes for increasing the rate of transfer hydrogenation to enhance the efficiency and/or yield of glycol include higher alcohols, such as ethanol, propanol, butanol, and other reagents that can serve as a source of hydrogen such as aldehydes.

Selection of the composition of catalysts in formoin condensation and transfer hydrogenation steps of the present processes is important for enabling highly selective, efficient and reproducible processes for making glycol reaction products. In some embodiments, for example, the compositions of the N-heterocyclic carbene catalyst and the transition metal hydrogen-transfer catalyst are selected to generate high yields of ethylene glycol with a reduction of reaction byproducts such as glycolaldehyde acetal.

N-heterocyclic carbene catalysts useful in some processes of the present invention are capable of catalysis of the self condensation of formaldehyde, for example to generate a glycoladehyde reaction product. In an embodiment, the N-heterocyclic carbene catalyst is a triazole-based catalyst or an imidazole-based catalyst. Some N-heterocyclic carbene catalysts useful in the present processes, for example, include a carbene or adduct thereof derived from a triazolium salt or an imidazolium salt. In an embodiment, the N-heterocyclic carbene catalyst is one or more compounds selected from the groups consisting of: a triazole carbene, an adduct of a triazole carbene, an imidazole carbene and an adduct of an imidazole carbene. N-heterocyclic carbene catalysts of the present invention can be generated in situ by dissolution of an appropriate catalyst precursor, such as a triazolium salt or an imidazolium salt in an appropriate solvent. In addition, N-heterocyclic carbene catalysts of the present invention, can be generated in situ via chemical reaction(s) of a catalyst precursor, for example, by dissociation reaction(s) and/or deprotonation by reaction(s) involving a reagent (e.g., a base) and a triazolium salt, an imidazolium salt or complex, adduct or derivative thereof. The molar ratio of N-heterocyclic carbene catalyst to formaldehyde provided to the reactor, is optionally in the range 0.05:100 to 20:100, and preferentially for some applications 1:100 to 5:100.

In a process of the present invention, the N-heterocyclic carbene catalyst is a triazole carbene, an imidazole carbene or an adduct of these. In an embodiment, for example, the N-heterocyclic carbene catalyst has the formula:

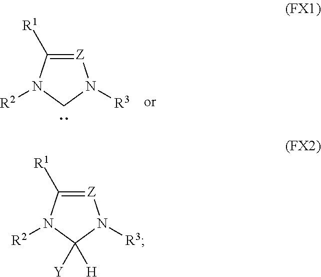

wherein each of R¹, R², R³ and R⁴ is independently a hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-₂₀ alkylsulfonyl or C₁-C₂₀ alkylsulfinyl; wherein Y is C₁-C₂₀ alkoxy or CCl₃; and wherein Z is CR⁴ or N. In an embodiment, the N-heterocyclic carbene catalyst has the formula (FX1) or (FX2) and at least of R¹, R², R³ and R⁴ is phenyl or C₅-C₂₀ aryl.

In an embodiment, for example, the N-heterocyclic carbene catalyst has the formula:

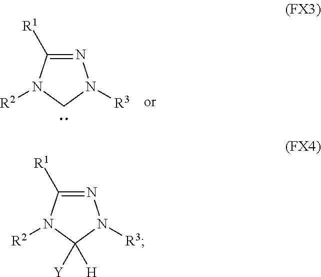

wherein each of R¹, R², and R³ is independently hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl; and wherein Y is a C₁-C₂₀ alkoxy or CCl₃. In an embodiment, the N-heterocyclic carbene catalyst has the formula (FX3) or (FX4) and at least of R¹, R², and R³ is phenyl or C₅-C₂₀ aryl. In an embodiment, the N-heterocyclic carbene catalyst has the formula (FX4) and Y is CH₃O.

In a process of the present invention particularly useful for production of ethylene glycol from formaldehyde, the N-heterocyclic carbene catalyst is Enders carbene or an adduct thereof. In an embodiment, for example, the N-heterocyclic carbene catalyst has the formula:

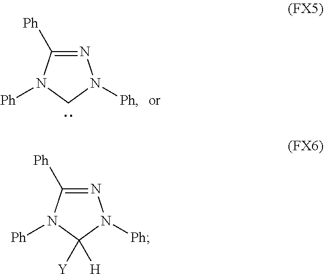

wherein Y is a C₁-C₂₀ alkoxy or CCl₃, and; wherein Ph is a phenyl group.

In an embodiment, for example, the N-heterocyclic carbene catalyst is Enders carbene as its methanol adduct. In an embodiment, the N-heterocyclic carbene catalyst is Enders carbene as its methanol adduct having the formula (FX7):

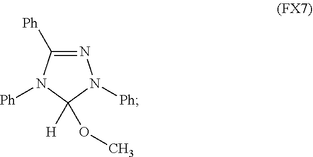

wherein Ph is a phenyl group. Use of Enders carbene as its methanol adduct for N-heterocyclic carbene catalysts in the present invention is particularly beneficial for selective production of ethylene glycol at high yields.

In an embodiment, for example, the N-heterocyclic carbene catalyst has the formula:

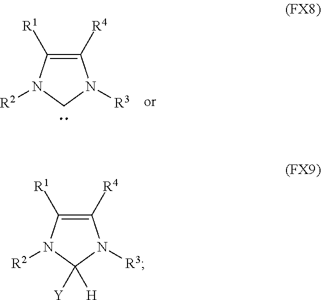

wherein each of R¹, R², R³ and R⁴ is independently hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl; and wherein Y is a C₁-C₂₀ alkoxy or CCl₃. In an embodiment, the N-heterocyclic carbene catalyst has the formula (FX8) or (FX9) and at least of R¹, R², R³ and R⁴ is phenyl or C₅-C₂₀ aryl. In an embodiment, the N-heterocyclic carbene catalyst has the formula (FX9) and Y is CH₃O. In an embodiment, the N-heterocyclic carbene catalyst has the formula (FX8) or (FX9) and at least of R¹, R², R³ and R⁴ is phenyl or C₅-C₂₀ aryl.

Transition metal hydrogen-transfer catalysts useful in some processes of the present invention are capable of effective catalysis of the transfer hydrogenation of reaction products generated by formoin condensation. In an embodiment, for example, a transition metal hydrogen-transfer catalyst is provided that is effective at catalyzing the transfer hydrogenation of glycoladehyde generated by formoin condensation to produce an ethylene glycol primary reaction product. Useful transition metal hydrogen-transfer catalysts include transition metal hydrides or halides (e.g., chlorides) or compounds derived from a transition metal hydride or a transition metal halide. Transition metal hydrogen-transfer catalysts may be generated in situ by dissolution of a catalyst precursor in a solvent and/or reaction of one or more catalyst precursors. In an embodiment, for example, the transition metal hydrogen-transfer catalyst is generated by reaction between a reagent(e.g., a base) and a transition metal complex, such as a transition metal hydride or halide. The molar ratio of transition metal hydrogen-transfer catalyst provided to the reactor to formaldehyde provided to the reactor, is optionally in the range 0.05:100 to 20:100, and preferentially for some applications 1:100 to 5:100.

In an embodiment, the transition metal hydrogen-transfer catalyst is one or more compounds selected from the group consisting of: RuCl₂(PPh₃)₃, RuH₂(PPh₃)₃, RuH₂(PPh₃)₄, RuH(OAc)(PPh₃)₃, Ru(OAc)₂(PPh₃)₃ (p-cymene)Ru(dpen)Cl (Noyori's catalyst), Cp*Ir(dpen)Cl, Cp*Ru(OMe)₂, [Cp*IrCl₂]₂, and Shvo's catalyst, or a compound derived from any of these. Use of Shvo's catalyst (C₆₂H₄₂O₆Ru₂; MW 1085.13; Tetracarbonyl-μ-hydro[(1,2,3,4,5-H)-1-hydroxylato-2,3,4,5-tetraphenyl-2,4-cyclopentadien-1-yl][(1,2,3,4,5-H)-1-hydroxy-2,3,4,5-tetraphenyl-2,4-cyclopentadien-1-yl]diruthenium) for transition metal hydrogen-transfer catalysts in the present processes is particularly beneficial for selective production of ethylene glycol at high yields. In some embodiments, a transition metal hydrogen-transfer catalyst selected from the group of RuCl₂(PPh₃)₃, RuH₂(PPh₃)₃, RuH₂(PPh₃)₄, RuH(OAc)(PPh₃)₃, Ru(OAc)₂(PPh₃)₃ is used for production of ethylene glycol.

In an embodiment, the transition metal hydrogen-transfer catalyst has the formula:

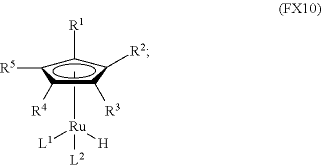

wherein each of R¹, R², R³, R⁴ and R⁵ is independently a hydrogen, hydroxyl, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂ alkoxycarbonyl, C₁-C₂₀ alkylthio, C₁-C₂₀ alkylsulfonyl or C₁-C₂₀ alkylsulfinyl; wherein each of L¹ and L² is independently CO or PR⁶₃; and wherein each R⁶ is independently a phenyl, cyclopentadiene, C₅-C₂₀ aryl, C₁-C₂₀ alkyl, or hydrogen. In an embodiment, the transition metal hydrogen-transfer catalyst has the formula (FX10) and at least one or R¹, R², R³, R⁴ and R⁵ is hydroxyl. In an embodiment, the transition metal hydrogen-transfer catalyst has the formula (FX10) and at least one of L¹ and L² is CO. In an embodiment, the transition metal hydrogen-transfer catalyst has the formula (FX10) and at least one of L¹ and L² is PPh₃. In an embodiment, the transition metal hydrogen-transfer catalyst has the formula (FX10) and L¹ is CO, L² is PPh₃, and R² is hydroxyl.

In an embodiment, the transition metal hydrogen-transfer catalyst has the formula:

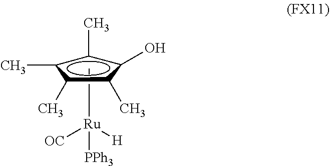

Process steps of the present invention may be carried out in a range of solvents and in the presence of a range of additional reagents and other additives. The formoin condensation and transfer hydrogenation steps of the present synthetic process can be carried out in the same or different solvents and/or in the presence of the same or different additional reagents or additives. In some embodiments, the process steps of the present invention are carried out in tetrahydrofuran (THF), as formoin condensation proceeds at fast rates in this solvent thereby providing an efficient synthetic pathway to ethylene glycol. Tetrahydrofuran (THF) also provides for good solubility of reaction products and intermediates involved in the present synthetic processes, thereby resulting in production of glycol at high yields. Other solvents useful in the present invention include any solvent that will dissolve the initial formaldehyde reagent, including, but not limited to, ethereal mixture, esters, and alcohols. Use of cyclic ethers solvents, such as dioxane and THF, are preferred for some applications for the present processes.

In an embodiment, the present invention provides a process for making a glycol from formaldehyde, wherein the glycol is ethylene glycol, propylene glycol or a mixture of ethylene glycol and propylene glycol. In an embodiment, the present invention provides a one pot synthesis process for producing ethylene glycol from formaldehyde. In an embodiment, the steps of contacting formaldehyde with the N-heterocyclic carbene catalyst and providing the transition metal hydrogen-transfer catalyst in contact with at least a portion of the one or more reaction products are carried out sequentially. In an embodiment, the present process further comprises the steps of: (i) contacting the formaldehyde and the N-heterocyclic carbene catalyst in a solvent, thereby generating the reaction products; and (ii) providing the transition metal hydrogen-transfer catalyst to the solvent containing the reaction products. In an embodiment, the present process further comprises the steps: (i) providing a mixture of formaldehyde and methanol; (ii) adding the N-heterocyclic carbene catalyst to the mixture; and (iii) adding the transition metal hydrogen-transfer catalyst to the mixture.

Formaldehyde may be provided in gas form or in the form of paraformaldehyde. In some embodiments, formaldehyde is provided as a mixture in the reactor, for example as a mixture with methanol and/or other a solvent(s) and/or additive(s). In processes of the present invention, the N-heterocyclic carbene catalyst and/or transition metal hydrogen-transfer catalyst can be provided to the solvent prior to, during, or after the addition of formaldehyde.

The processes of the present invention may further comprise the step of providing one or more additives to the reactor. In addition to formaldehyde, there may also be introduced to the reactor additional feedstock components, such as bases and/or metal salts such as borates. The present invention includes processes further comprising the step of providing a base for formoin condensation and/or transfer hydrogenation steps. Certain additional feedstock components may advantageously act as initiators for the formoin condensation and/or transfer hydrogenation reactions of the present processes. In the context of the present description, the term “initiator” refers to an additive that causes a chemical reaction or series of chemical reactions to take place and/or enhances the rate of a chemical reaction or series of chemical reactions.

In some embodiments, the processes of the present invention are performed at a temperature in the range of 20 degrees Celsius to 65 degrees Celsius. In some processes, one or more of the steps of the present invention are carried out at a temperature greater than 70 degrees Celsius for a time period selected from the range of 1 minute to 600 minutes. In some embodiments, one or more of the process steps of the present invention are carried out in an inert atmosphere, for example under argon gas.

The processes of the present invention may be performed as a batch or as a continuous process. When operated as a continuous process, formaldehyde reactant may be introduced continuously, together or separately, into the reactor and the glycol reaction product may be continuously removed from the reactor.

As will be understood by those having skill in the art, a range of reactors may be used in the present processes. In an embodiment, for example, the process of the present invention is performed in a reactor which is suitably an adiabatic reactor or a reactor configured with heat-removal system(s) or mechanism(s) such as cooling coils which may remove, for example, up to 20% of the heat of reaction.

Without wishing to be bound by any particular theory, there can be discussion herein of beliefs or understandings of underlying principles or mechanisms relating to the invention. It is recognized that regardless of the ultimate correctness of any explanation or hypothesis, an embodiment of the invention can nonetheless be operative and useful.

### BRIEF DESCRIPTION OF THE DRAWINGS

FIG. 1 provides a scheme showing the transformation of formaldehyde to glycolaldehyde using a triazolium salt catalyst.

FIG. 2 provides individual reactions and the net process showing a coupled catalytic formoin condensation and transfer hydrogenation processes of the present invention.

FIG. 3 provides a chromatogram showing reaction products generated in the transfer hydrogenation step of the present processes. The chromatogram show peaks for the ethylene glycol reaction product, as well as glycolaldehyde acetal and methylglycolate reaction byproducts.

FIG. 4 provides a scheme showing the formation of methyl glycolate. The hydrogen produced in this transformation may be transferred by the metal catalyst (e.g., Shvo's catalyst) and used for reduction of glycolaldehyde in the present processes.

FIG. 5 shows the reaction scheme and observed yields of ethylene glycol for a transfer hydrogenation process step of the present invention. As shown in FIG. 5, yields of ethylene glycol from transfer hydrogenation reaction of glycolaldehyde as large as 50% are achievable.

FIG. 6 provides the reaction scheme for transfer hydrogenation in the presence of a base to generate ethylene glycol.

FIG. 7 provides a scheme showing transformation of formaldehyde to ethylene glycol. As shown in this figure, a first step involves generation of glycolaldehyde via formoin condensation catalyzed by Enders carbene as its methanol adduct and subsequent transfer hydrogenation by reaction with methanol in the presence of Shvo's catalyst. Yields of the ethylene glycol reaction product and reaction byproducts are provided for reaction conditions in toluene and THF solvents.

FIG. 8 provides a scheme showing the reaction of formaldehyde and methanol in the presence of Enders carbene as its methanol adduct and Shov's Catalyst in toluene solvent to generate methyl formate as the exclusive reaction product.

### DETAILED DESCRIPTION OF THE INVENTION

Referring to the drawings, like numerals indicate like elements and the same number appearing in more than one drawing refers to the same element. In general the terms and phrases used herein have their art-recognized meaning, which can be found by reference to standard texts, journal references and contexts known to those skilled in the art. The following definitions are provided to clarify their specific use in the context of the invention.

As used herein, a N-heterocyclic carbene catalyst refers to a cyclic, nitrogen-containing carbene or complex, adduct, derivative or reaction product thereof. N-heterocyclic carbene catalysts useful in aspects of the present invention are capable of catalyzing self condensation of formaldehyde, for example via formoin condensation to generate a glycoladehyde reaction product. In an embodiment, the N-heterocyclic carbene catalyst is a triazole-based catalyst or an imidazole-based catalyst, such as a carbene or adduct, complex, or ylid thereof derived from a triazolium salt or an imidazolium salt. N-heterocyclic carbene catalysts of the present invention can be generated in situ by dissolution of a catalyst precursor, such as a triazolium salt or an imidazolium salt in an appropriate solvent. In addition, N-heterocyclic carbene catalysts of the present invention, can be generated in situ via chemical reaction of a catalyst precursor, for example, by dissociation reaction(s) and/or deprotonation by reaction(s) with a base involving a triazolium salt, an imidazolium salt or complex, adduct or derivative thereof. In some processes of the present invention, the N-heterocyclic carbene catalyst is Enders carbene and/or an adduct thereof, such as Enders carbene as its methanol or chloroform adducts.

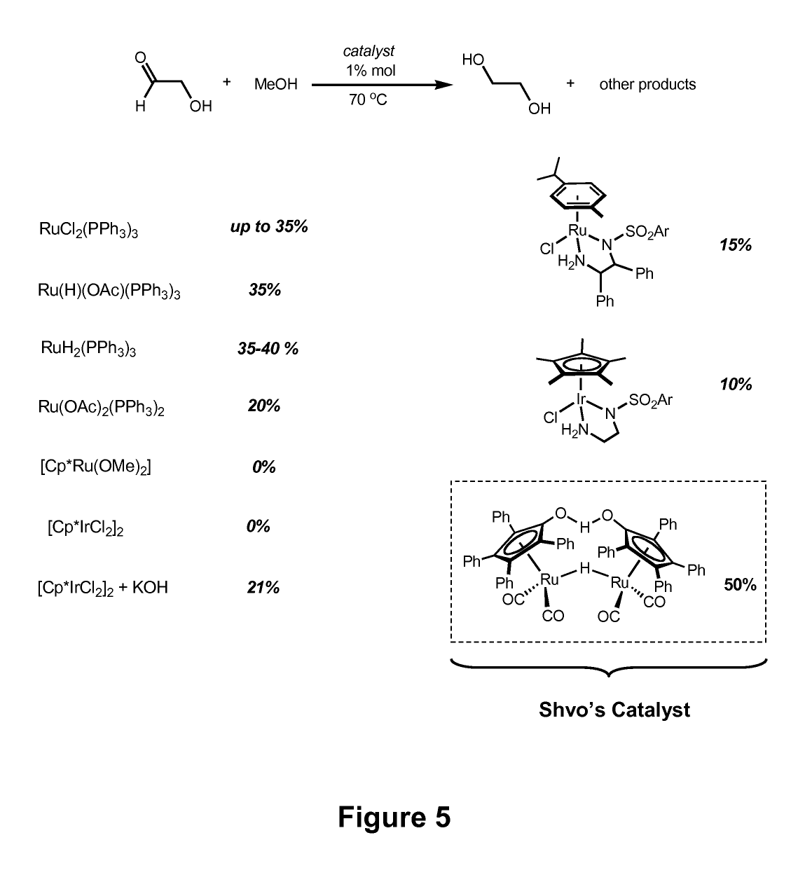

As used herein, a transition metal hydrogen-transfer catalyst refers to a compound for catalyzing a transfer hydrogenation reaction, for example, a transfer hydrogenation reaction of a reaction product from formoin condensation that results in the production of a glycol, such as ethylene glycol. In an embodiment, for example, a transition metal hydrogen-transfer catalyst of the present invention is effective at catalyzing the transfer hydrogenation of glycoladehyde generated by formoin condensation to produce an ethylene glycol reaction product. Useful transition metal hydrogen-transfer catalysts include transition metal hydrides or compounds derived from transition metal hydrides and transition metal halides. Transition metal hydrogen-transfer catalysts may be generated in situ by dissolution of a catalyst precursor in a solvent and/or reaction of one or more catalyst precursors. Use of Shvo's catalyst (C₆₂H₄₂O₆Ru₂; MW 1085.13; Tetracarbonyl-μ-hydro[(1,2,3,4,5-H)-1-hydroxylato-2,3,4,5-tetraphenyl-2,4-cyclopentadien-1-yl][(1,2,3,4,5-H)-1-hydroxy-2,3,4,5-tetraphenyl-2,4-cyclopentadien-1-yl]diruthenium) for transition metal hydrogen-transfer catalysts in the present invention is particularly beneficial for selective production of ethylene glycol at high yields. FIG. 5 provides the structure of a form of Shvo's catalyst useful in the present processes.

Alkyl groups include straight-chain, branched and cyclic (e.g., cycloalkyl) alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. Cyclic alkyl groups include those having one or more rings. Cyclic alkyl groups include those having a 3-, 4-, 5-, 6-, 7-, 8-, 9- or 10-member carbon ring and particularly those having a 3-, 4-, 5-, 6-, or 7-member ring. The carbon rings in cyclic alkyl groups can also carry alkyl groups. Cyclic alkyl groups can include bicyclic and tricyclic alkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxyl group is an alkyl group linked to oxygen and can be represented by the formula R—O.

Alkenyl groups include straight-chain, branched and cyclic (e.g., cycloalkenyl) alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cyclic alkenyl groups include those having one or more rings. Cyclic alkenyl groups include those in which a double bond is in the ring or in an alkenyl group attached to a ring. Cyclic alkenyl groups include those having a 3-, 4-, 5-, 6-, 7-, 8-, 9- or 10-member carbon ring and particularly those having a 3-, 4-, 5-, 6- or 7-member ring. The carbon rings in cyclic alkenyl groups can also carry alkyl groups. Cyclic alkenyl groups can include bicyclic and tricyclic alkyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogens replaced with one or more fluorine atoms.

Aryl groups include groups having one or more 5- or 6-member aromatic or heteroaromatic rings. Aryl groups can contain one or more fused aromatic rings. Heteroaromatic rings can include one or more N, O, or S atoms in the ring. Heteroaromatic rings can include those with one, two or three N, those with one or two O, and those with one or two S, or combinations of one or two or three N, O or S. Aryl groups are optionally substituted. Substituted aryl groups include among others those which are substituted with alkyl or alkenyl groups, which groups in turn can be optionally substituted. Specific aryl groups include phenyl groups, biphenyl groups, pyridinyl groups, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl having one or more hydrogens replaced with one or more fluorine atoms, chlorine atoms, bromine atoms and/or iodine atoms.

Optional substitution of any alkyl, alkenyl, and aryl groups includes substitution with one or more of the following substituents: halogens, —CN, —COOR, —OR, —COR, —OCOOR, —CON(R)₂, —OCON(R)₂, —N(R)₂, —NO₂, —SR, —SO₂R, —SO₂N(R)₂ or —SOR groups. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

The term “alkoxy” as used herein refers to an alkyl group bound through a single, terminal ether linkage. An alkoxy group may be represented as: O-alkyl (e.g. OCH₃) where alkyl is as defined above.

Optional substituents for alkyl, alkenyl and aryl groups include among others:
—COOR where R is a hydrogen or an alkyl group or an aryl group and more specifically where R is methyl, ethyl, propyl, butyl, or phenyl groups all of which are optionally substituted;
—COR where R is a hydrogen, or an alkyl group or an aryl groups and more specifically where R is methyl, ethyl, propyl, butyl, or phenyl groups all of which groups are optionally substituted;
—CON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—OCON(R)₂ where each R, independently of each other R, is a hydrogen or an alkyl group or an aryl group and more specifically where R is methyl, ethyl, propyl, butyl, or phenyl groups all of which groups are optionally substituted; R and R can form a ring which may contain one or more double bonds;
—N(R)₂ where each R, independently of each other R, is a hydrogen, or an alkyl group, acyl group or an aryl group and more specifically where R is methyl, ethyl, propyl, butyl, or phenyl or acetyl groups all of which are optionally substituted; or R and R can form a ring which may contain one or more double bonds.
—SR, —SO₂R, or —SOR where R is an alkyl group or an aryl groups and more specifically where R is methyl, ethyl, propyl, butyl, phenyl groups all of which are optionally substituted; for —SR, R can be hydrogen;
—OCOORR where R is an alkyl group or an aryl groups;
—SO₂N(R)₂ where R is a hydrogen, an alkyl group, or an aryl group and R and R can form a ring;
—OR where R═H, alkyl, aryl, or acyl; for example, R can be an acyl yielding —OCOR* where R* is a hydrogen or an alkyl group or an aryl group and more specifically where R* is methyl, ethyl, propyl, butyl, or phenyl groups all of which groups are optionally substituted;

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups, and methoxyphenyl groups, particularly 4-methoxyphenyl groups.

As used herein, the term “alkylene” refers to a divalent radical derived from an alkyl group as defined herein. Alkylene groups in some embodiments function as bridging and/or spacer groups in the present compositions.

As used herein, the term “cycloalkylene” refers to a divalent radical derived from a cycloalkyl group as defined herein. Cycloalkylene groups in some embodiments function as bridging and/or spacer groups in the present compositions.

As used herein, the term “alkenylene” refers to a divalent radical derived from an alkenyl group as defined herein. Alkenylene groups in some embodiments function as bridging and/or spacer groups in the present compositions.

As used herein, the term “cylcoalkenylene” refers to a divalent radical derived from a cylcoalkenyl group as defined herein. Cycloalkenylene groups in some embodiments function as bridging and/or spacer groups in the present compositions.

As used herein, the term “alkynylene” refers to a divalent radical derived from an alkynyl group as defined herein. Alkynylene groups in some embodiments function as bridging and/or spacer groups in the present compositions.

The following abbreviations are used throughout the present description. Cp refers to a cyclopentadiene group. Ph refers to a phenyl group. Cy refers to a cylcoalkyl group. OAc refers to an acetoxy group.

As to any of the above groups which contain one or more substituents, it is understood, that such groups do not contain any substitution or substitution patterns which are sterically impractical and/or synthetically non-feasible. In addition, the compounds of this invention include all stereochemical isomers arising from the substitution of these compounds.

The so-called Formose reaction is a method for the synthesis of carbohydrates directly from formaldehyde. The reaction is usually catalyzed by a number of species including calcium hydroxide, cyanide and a variety of imidazolium and thiazolium salts.¹ Although potentially useful, this process is typically unselective and sometimes irreproducible, yielding various types of hydrocarbons ranging from C₂ to C₇.²

Furthermore, the reaction appears sensitive to even small changes in experimental conditions making it difficult to predict. The first step in this reaction is the self-condensation of formaldehyde to form glycoladehyde, the so-called formoin condensation. Glycolaldehyde undergoes subsequent reactions to form higher carbohydrates.³

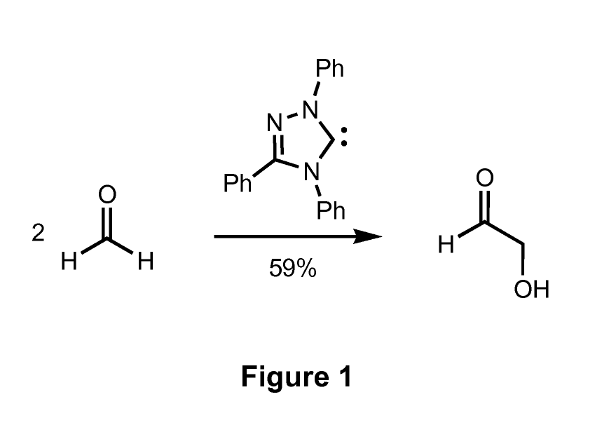

N-heterocyclic carbenes (NHC) have received much attention in recent years as ligands for transition metals, but also as organocatalysts for a number of reactions due to their ability to form acylanion equivalents.¹ N-heterocyclic carbenes have also been implicated as direct intermediates in the formose reaction catalyzed by imidazolium and thiazolium salts, showing significant levels of selectivity for lower number carbon fragments. For instance, it has been found that formaldehyde can be converted into dihydroxyacetone in up to 70% yield, using a combination of ethylbenzothiazolium bromide and trimethylamine.⁴ Notably, there is only one report in the literature describing selectivity for the formoin condensation. Enders, Teles and co-workers have shown that using the triazolium salt shown in FIG. 1, yields of up to 59% of glycoldehyde are achievable.⁵

This transformation is of practical interest since glycolaldehyde could be directly hydrogenated to ethylene glycol, a commodity of significant industrial interest. Although ethylene glycol is industrially prepared from the hydration of ethylene oxide, protocols for its synthesis via hydrogenation of glycolaldehyde with H₂ have been reported in the patent literature.⁶ Ideally, however, such reduction could be carried out by a mild transfer hydrogenation process using cheap feedstocks such as methanol.

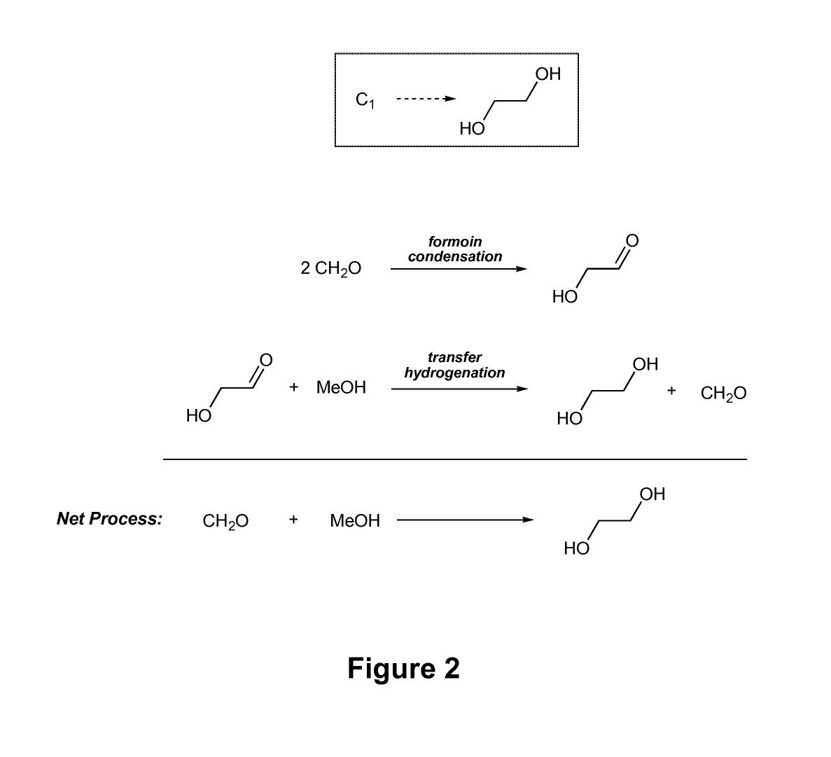

The present invention provides processes for the synthesis of ethylene glycol by a tandem formoin condensation/transfer hydrogenation protocol. FIG. 2 provides individual reactions and the net process showing a coupled catalytic formoin condensation and transfer hydrogenation process of the present invention. In an embodiment, the formoin condensation is carried out using Enders carbene as its methanol adduct. The subsequent transfer hydrogenation step is performed using a variety of transition-metal complexes and a source of hydrogen, such as methanol, optionally provided a solvent.

### Example 1

### Generation of Ethylene Glycol via Catalyzed Formoin Condensation and Transfer Hydrogenation

To demonstrate the capability of the present synthetic processes to selectively and efficiently generate ethylene glycol in high yields, formoin condensation reaction using a triazole-based catalyst and transfer hydrogenation of glycolaldehyde by reaction with methanol in the presence of a transition metal hydrogen-transfer catalyst were studied. The results of this Example demonstrate formation of ethylene glycol with yields of 18-20% are achievable using the present processes.

Ender's carbene and its methanol adduct were prepared by well known literature procedures. Metal catalysts RuCl₂(PPh₃)₃, RuH₂(PPh₃)₃, RuH₂(PPh₃)₄, RuH(OAc)(PPh₃)₃, Ru(OAc)₂PPh₃)₃ (p-cymene)Ru(dpen)Cl (Noyori's catalyst), and Cp*Ir(dpen)Cl were also prepared by known methods. Cp*Ru(OMe)₂, [Cp*IrCl₂]₂, and Shvo's catalyst were purchased from Strem and used as received. Formaldehyde (95%, powder) and glycoladehyde were obtained from Aldrich and used as received. MeOH (Aldrich) was dried using known protocols.

1.a. Transfer Hydrogenation Experiments

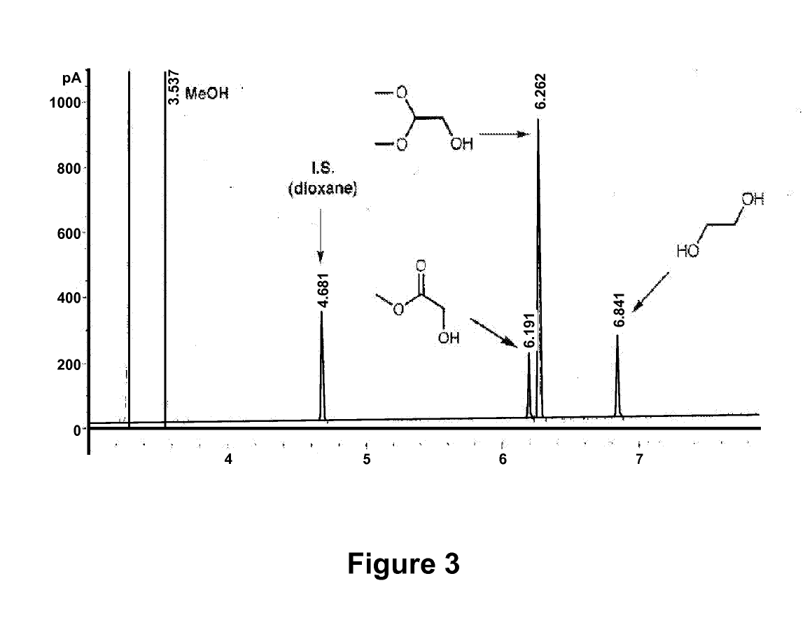

In a typical experiment, 120 mg (2.0 mmol based on monomer) of glycolaldehyde were weighed into a glass bomb fitted with a Teflon valve. The bomb was evacuated and refilled with argon and metal catalyst (0.02 mmol) was added under a counterflow of argon. The bomb was then fitted with a rubber septa and 5 mL of dry methanol were injected followed by 30 μl of dioxane (internal standard). The septa was replaced by a Teflon valve and the system was heated in an oil bath at 70° C. for 2-5 hrs. The system was cooled and a sample of this mixture was then syringed out and passed through a Stratopheres™ plug to remove the transition metal. The sample was analyzed by GC and ethylene glycol quantified. Glycolaldehyde acetal and methylglycolate were obtained as the major by-products along with ethylene glycol. Yields of ethylene glycol ranging from 0-40% using RuCl₂(PPh₃)₃, RuH₂(PPh₃)₃, RuH₂(PPh₃)₄, RuH(OAc)(PPh₃)₃, Ru(OAc)₂(PPh₃)₃, Cp*Ir(dpen)Cl, Cp*Ru(OMe)₂ and [Cp*IrCl₂]₂ were typically obtained. Substantial amounts of acetal were obtained primarily when RuCl₂(PPh₃)₃, and particularly [Cp*IrCl₂]₂, were used. A typical chromatogram is shown in FIG. 3 showing the production of ethylene glycol.

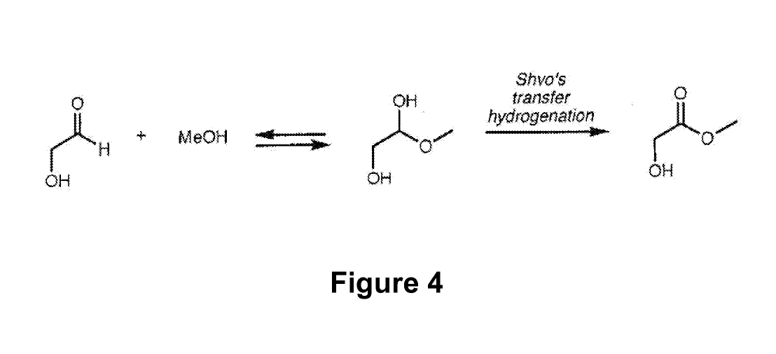

The transfer hydrogenation step was studied first in a separate set of experiments. Ethylene glycol was produced in varied yields. Under some experimental conditions, significant amounts of glycolaldehyde dimethylacetal and methyl glycolate were also formed as by-products. While acetal formation can be completely suppressed depending on the transition-metal complex used, formation of methyl glycolate is unavoidable and appears intrinsic to the overall process. FIG. 4 provides a scheme showing the formation of methyl glycolate. The hydrogen produced in this transformation may be transferred by the metal catalyst (e.g., Shvo's catalyst) and used for reduction of glycolaldehyde in the present processes. FIG. 5 shows the reaction scheme and observed yields of ethylene glycol for a transfer hydrogenation process step of the present invention. As shown in FIG. 5, yields of ethylene glycol from the transfer hydrogenation reaction of glycolaldehyde as large as 50% are achievable. Table 1 summarizes the observed yields of ethylene glycol for a number transition metal transfer hydrogen catalysts.

TABLE 1
Ethylene Glycol Yields for Transition Metal Transfer Hydrogen Catalysts.

| | Yield of |
|---|---|
| Transition Metal | Ethylene |
| Hydrogen-Transfer Catalyst | Glycol |
| RuCl₂(PPh₃)₃ | Up to 35% |
| Ru(H)(OAc)(PPh₃)₃ | 35% |
| RuH₂(PPh₃)₃ | 35-40% |
| Ru(OAc)₂(PPh₃)₂ | 20% |
| [Cp*Ru(OMe)₂] | 0% |
| [Cp*IrCl₂]₂ | 0% |
| [Cp*IrCl₂]₂ + KOH | 21% |
| 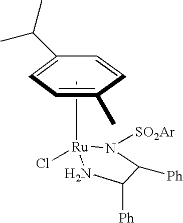 | 15% |
| 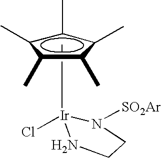 | 10% |
| 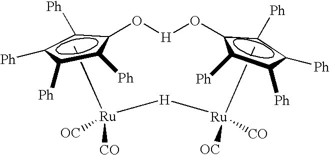 | 50% |

The hydrogen produced in this transformation is transferred by the metal catalyst and used for the reduction of glycolaldehyde. Thus, the maximum theoretical yield for ethylene glycol is expected to be 50%.

An overall formoin condensation/transfer hydrogenation sequence can be carried out in one pot by sequential addition of catalysts (e.g., carbene and Shvo's complex) affording an overall yield of ethylene glycol of ˜18-20%. Solvent plays an important role due to competing pathways. In toluene the formoin condensation is slow yielding methyl formate as the exclusive product upon addition of metal catalyst in methanol. In THF the formoin condensation proceeds faster, providing good yields of glycolaldehyde as judged by the high conversion to ethylene glycol upon transfer hydrogenation in methanol. This medium also provides good solubility for all the intermediates involved.

1.b. Effect of Base

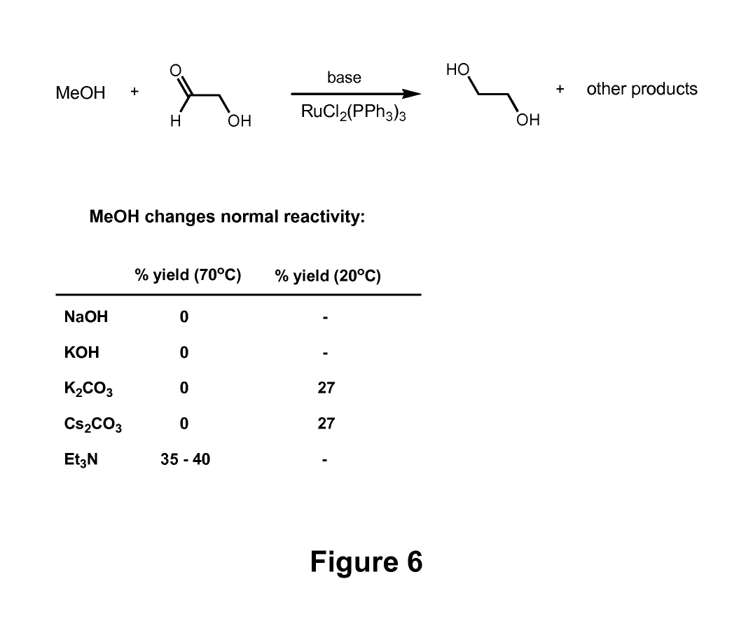

The use of a base to generate metal hydrides in situ from metal chlorides and methanol was explored. FIG. 6 provides the reaction scheme for transfer hydrogenation in the presence of a base to generate ethylene glycol. In a standard procedure, 120 mg (2.0 mmol based on monomer) of glycolaldehyde were weighed into a glass bomb fitted with a Teflon valve. The bomb was evacuated and refilled with argon and metal catalyst (0.02 mmol) followed by base (et₃N, NaOH, KOH, Cs₂CO₃, Na₂CO₃ or K₂CO₃, 0.1 mmol) was added under a counterflow of argon. The bomb was then fitted with a rubber septa and 5 mL of dry methanol were injected followed by 30 μL of dioxane (internal standard). The septa was replaced by a Teflon valve and the system was heated in an oil bath at 70° C. for 2-5 hrs. The system was cooled and a sample of this mixture was then syringed and passed through a Stratopheres™ plug to remove the transition metal. The sample was analyzed by GC and ethylene glycol quantified. High yields were observed for certain bases, especially at lower temperatures, as shown in Table 2. At room temperature for some reaction conditions the amount of side products diminishes substantially, but ethylene glycol production is decreases after 3 hours and observed yields are lower.

TABLE 2
Ethylene Glycol Yields for Reaction Conditions in the Presence of a Base

| | Ethylene glycol | Ethylene glycol |
|---|---|---|
| | % yield (70° C.) | % yield (20° C.) |
| NaOH | 0 | — |
| KOH | 0 | — |
| K₂CO₃ | 0 | 27 |
| Cs₂CO₃ | 0 | 27 |
| Et₃N | 35-40 | — |

1.c. Use of Shvo's Catalyst

120 mg (2.0 mmol based on monomer) of glycolaldehyde were weighed into a glass bomb fitted with a Teflon valve. The bomb was evacuated and refilled with argon and Shvo's catalyst (0.02 mmol) was added under a counterflow of argon. The bomb was then fitted with a rubber septa and 4 mL of dry toluene and 1 mL of methanol were injected followed by 30 μL of dioxane (internal standard). The septa was replaced by a Teflon valve and the system was heated in an oil bath at 70° C. for 5 hrs. The sample was analyzed by GC and ethylene glycol quantified. A 1:1 mixture of ethylene glycol and methyl glycolate was obtained quantitatively. Glycolaldehyde acetal formation was completely suppressed. Use of Shvo's catalyst results in a yield of ethylene glycol of 50% under some reaction conditions.

1.d. Use of Shvo's Catalyst in the Presence of Water

As a potential way to avoid methyl glycolate formation, known amounts of water were added to the system to supress the methanol-glycolaldehyde hemiacetal equilibrium. 120 mg (2.0 mmol based on monomer) of glycolaldehyde were weighed into a glass bomb fitted with a Teflon valve. The bomb was evacuated and refilled with argon and Shvo's catalyst (0.02 mmol) was added under a counterflow of argon. The bomb was then fitted with a rubber septa and 0.82 mL (20 mmol) of methanol, 0.45 mL (25 mmol) of degassed water and 3.5 mL of THF were injected into the system. Dioxane (30 μL) was added as an internal standard. The septa was replaced by a Teflon valve and the system was heated in an oil bath at 70° C. for 5 hrs. The sample was analyzed by GC and ethylene glycol quantified. Methyl glycolate formation was not suppressed and the results were similar to those described above in Section 1.c.

1.e. One Pot Synthesis of Ethylene Glycol from Formaldehyde

Under argon, 240 mg (8 mmol based on monomer) of paraformaldehyde and 13 mg (0.04 mmol) of Enders carbene (as its methanol adduct) were weighed into a glass bomb and the mixture dissolved in 2.2 mL of THF. The system was then heated at 70° C. Within 1-2 minutes, the insoluble paraformaldehyde is consumed to give a clear light green solution. After heating for 10 minutes, the flask was cooled at RT. Under argon, 22 mg of Shvo's catalyst (0.02 mmol), 2 mL of dry methanol and dioxane (30 μL) were added to the mixture and the flask heated again for 5 hrs. The sample was analyzed by GC and ethylene glycol quantified. The yields of ethylene glycol are typically ˜18-20%. Replacing THF for toluene affords no ethylene glycol.

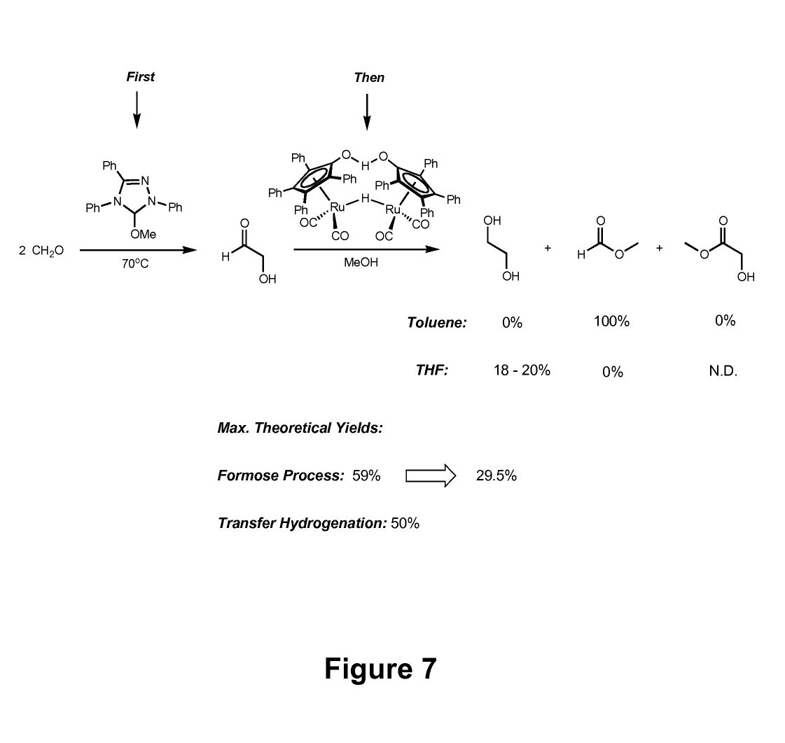

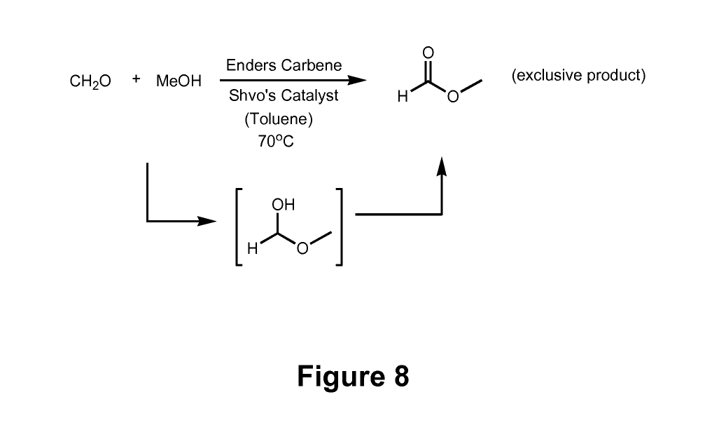

FIG. 7 provides an overview scheme showing transformation of formaldehyde to ethylene glycol. As shown in this figure, a first step involves generation of glycolaldehyde via formoin condensation catalyzed by Enders carbene as its methanol adduct and subsequent transfer hydrogenation by reaction with methanol in the presence of Shvo's catalyst. Yields of the ethylene glycol reaction product and reaction byproducts are provided for reaction in toluene and THF solvents. FIG. 8 provides a scheme showing the reaction of formaldehyde and methanol in the presence of Enders carbene as its methanol adduct and Shov's Catalyst in toluene solvent to generate methyl formate as the exclusive reaction product.

### Statements Regarding Incorporation by Reference and Variations

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Many of the molecules disclosed herein contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every formulation or combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or process steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

### REFERENCES

¹Enders, D.; Balensiefer, T, Acc. Chem. Res, 2004, 37, 534
²Niitsu, T.; Ito, M. M.; Inoue, H. J. Chem, Eng. Jpn, 1992, 25,480
³Storey, J. M, D.; Williamson, C. Tet. Lett. 2005, 46, 7337
⁴lnoue, S.; Matsumoto, T. J. Chem. Soc., Chem. Commun. 1983, 171
⁵Teles, J. H.; Melder, J-P.; Ebel, K.; Schneider, R.; Geher, E.; Harder, W.; Brode, S.; Enders, D.; Breuer, K.; Raabe, G. Helv. Chim. Acta 1996, 79, 61
⁶Costa, L. C. U.S. Pat. No. 4,321,414 (1982)

1. A process for the production of a glycol, said process comprising the steps of:
contacting formaldehyde with a N-heterocyclic carbene catalyst thereby generating one or more reaction products; and
providing a transition metal hydrogen-transfer catalyst in contact with at least a portion of said one or more reaction products, thereby producing said glycol.

2. The process of claim 1 wherein the transition metal hydrogen-transfer catalyst is provided in contact with said one or more reaction products in the presence of an alcohol.

3. The process of claim 2 wherein said alcohol is methanol.

4. The process of claim 1 wherein said N-heterocyclic carbene catalyst is a triazole-based catalyst or an imidazole-based catalyst.

5. The process of claim 1 wherein said N-heterocyclic carbene catalyst is a carbene or adduct thereof derived from a triazolium salt or an imidazolium salt.

6. The process of claim 1 wherein said N-heterocyclic carbene catalyst is one or more compounds selected from the groups consisting of: a triazole carbene, an adduct of a triazole carbene, an imidazole carbene and an adduct of an imidazole carbene.

7. The process of claim 1 wherein said N-heterocyclic carbene catalyst has the formula: 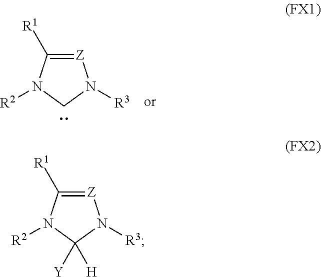
wherein each of R¹, R², R³ and R⁴ is independently a hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl; wherein Y is C₁-C₂₀ alkoxy or CCl₃, and wherein Z is CR⁴ or N.

8. The process of claim 1 wherein said N-heterocyclic carbene catalyst has the formula: 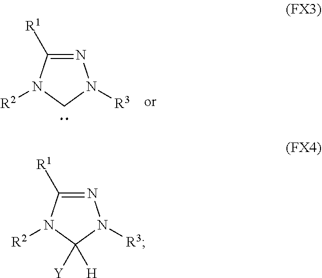
wherein each of R¹, R², and R³ is independently hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl; and wherein Y is a C₁-C₂₀ alkoxy or CCl₃.

9. The process of claim 1 wherein said N-heterocyclic carbene catalyst is Enders carbene having the formula: 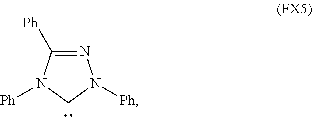
or an adduct thereof having the formula 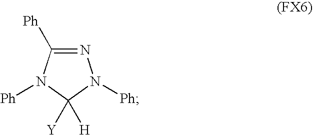
wherein Y is a C₁-C₂₀ alkoxy or CCl₃, and; wherein Ph is a phenyl.

10. The process of claim 1 wherein said N-heterocyclic carbene catalyst is Enders carbene as its methanol adduct having the formula (FX7): 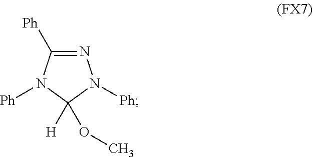
wherein Ph is a phenyl.

11. The process of claim 1 the N-heterocyclic carbene catalyst has the formula: 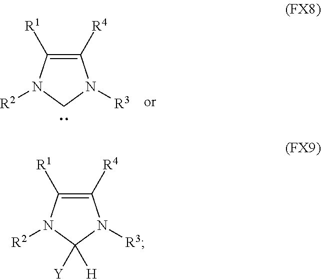
wherein each of R¹, R², R³ and R⁴ is independently hydrogen, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl; and wherein Y is a C₁-C₂₀ alkoxy or CCl₃.

12. The process of claim 1 wherein said transition metal hydrogen-transfer catalyst is a transition metal hydride or a transition metal chloride.

13. The process of claim 1 wherein said transition metal hydrogen-transfer catalyst is one or more compounds selected from the group consisting of: RuCl₂(PPh₃)₃, RuH₂(PPh₃)₃, RuH₂(PPh₃)₄, RuH(OAc)(PPh₃)₃, Ru(OAc)₂(PPh₃)₃(p-cymene)Ru(dpen)Cl, Cp*Ir(dpen)Cl, Cp*Ru(OMe)₂, [Cp*IrCl₂]₂, and Shvo's catalyst.

14. The process of claim 1 wherein said transition metal hydrogen-transfer catalyst is Shvo's catalyst.

15. The process of claim 1 wherein the transition metal hydrogen-transfer catalyst has the formula: 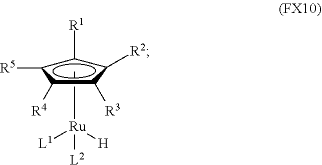
wherein each of R¹, R², R³, R⁴ and R⁵ is independently a hydrogen, hydroxyl, C₁-C₂₀ alkyl, C₅-C₂₀ aryl, C₁-C₂₀ acyl, C₂-C₂₀ alkenyl, C₂-C₂₀ alkynyl, C₁-C₂₀ carboxylate, C₁-C₂₀ alkoxy, C₂-C₂₀ alkenyloxy, C₂-C₂₀ alkynyloxy, C₅-C₂₀ aryloxy, C₂-C₂₀ alkoxycarbonyl, C₁-C₂₀ alkylthio, C₁-C₂₀ alkylsulfonyl or C₁-C₂₀ alkylsulfinyl;
wherein each of L¹ and L² is independently CO or PR⁶₃; and
wherein each R⁶ is independently a phenyl, cyclopentadiene, C₅-C₂₀ aryl, C₁-C₂₀ alkyl, or hydrogen.

16. The process of claim 15 wherein at least one or R¹, R², R³, R⁴ and R⁵ is hydroxyl.

17. The process of claim 15 wherein the transition metal hydrogen-transfer catalyst has the formula: 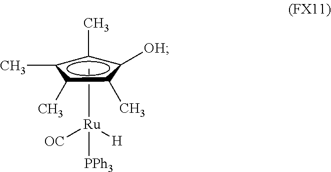
wherein Ph is phenyl.

18. The process of claim 15 wherein at least one of L¹ and L² is PPh₃, wherein Ph is phenyl.

19. The process of claim 15 wherein at least one of L¹ and L² is CO.

20. The process of claim 1 wherein the glycol is ethylene glycol, propylene glycol or a mixture of ethylene glycol and propylene glycol.

21. The process of claim 1 comprising a one pot synthesis process for producing ethylene glycol from formaldehyde.

22. The process of claim 1 wherein said steps of contacting formaldehyde with said N-heterocyclic carbene catalyst and providing said transition metal hydrogen-transfer catalyst in contact with at least a portion of said one or more reaction products are carried out sequentially.

23. The process of claim 1 wherein said step of contacting formaldehyde with said N-heterocyclic carbene catalyst initiates catalytic condensation of said formaldehyde to generate a glycolaldehyde reaction product.

24. The process of claim 23 wherein said step of providing said transition metal hydrogen-transfer catalyst in contact with at least a portion of said one or more reaction products initiates catalytic transfer hydrogenation of said glycolaldehyde reaction product to generate ethylene glycol.

25. The process of claim 1 further comprising the steps of:
contacting said formaldehyde and said N-heterocyclic carbene catalyst in a solvent, thereby generating said reaction products; and
providing said transition metal hydrogen-transfer catalyst to said solvent containing said reaction products.

26. The process of claim 1 further comprising:
providing a mixture of formaldehyde and methanol;
adding said N-heterocyclic carbene catalyst to said mixture; and
adding said transition metal hydrogen-transfer catalyst to said mixture.

27. The process of claim 1 wherein said step of contacting formaldehyde with said N-heterocyclic carbene catalyst, said step of providing said transition metal hydrogen-transfer catalyst in contact with at least a portion of said one or more reaction products or both of said steps of contacting formaldehyde with said N-heterocyclic carbene catalyst and providing said transition metal hydrogen-transfer catalyst in contact with at least a portion of said one or more reaction products is carried out in one or more solvents selected from the group consisting of a C₁-C₂₀ alcohol, ethereal mixture, an ester, a cyclic ethers solvents.

28. The process of claim 27 wherein said solvent is tetrahydrofuran or dioxane.

29. The process of claim 1 wherein said process is carried out in a reactor; wherein the molar ratio of N-heterocyclic carbene catalyst provided to the reactor to formaldehyde provided to the reactor is selected over the range 0.05:100 to 20:100.

30. The process of claim 1 wherein said process is carried out in a reactor; wherein the molar ratio of transition metal hydrogen-transfer catalyst provided to the reactor to formaldehyde provided to the reactor is selected over the range of the range 0.05:100 to 20:100.

31. The process of claim 1 wherein said process is carried out in a reactor; wherein the transition metal hydrogen-transfer catalyst is contacted with said formaldehyde or said one or more reaction products in the presence of an alcohol; and wherein the molar ratio of formaldehyde provided to the reactor to alcohol provided to the reactor is selected over the range of 0.01 to 0.7.

32. The process of claim 1 wherein said step of contacting formaldehyde with said N-heterocyclic carbene catalyst is carried out at a temperature selected over the range of 20 degrees Celsius to 65 degrees Celsius.

33. The process of claim 1 wherein said step of contacting formaldehyde with said N-heterocyclic carbene catalyst is carried out at a temperature greater than or equal to 70 degrees Celsius for a time period selected over the range of 1 minute to 600 minutes.

34. The process of claim 1 wherein said step of providing said transition metal hydrogen-transfer catalyst in contact with at least a portion of said one or more reaction products is carried out at a temperature selected over the range of 20 degrees Celsius to 65 degrees Celsius.

35. The process of claim 1 wherein said step of providing said transition metal hydrogen-transfer catalyst in contact with at least a portion of said one or more reaction products is carried out at a temperature greater than or equal to 70 degrees Celsius for a time period selected over the range of 1 minute to 600 minutes.

36. The process of claim 1 wherein said step of providing a transition metal hydrogen-transfer catalyst in contact with at least a portion of said one or more reaction products is carried out under argon.

37. The process of claim 1 wherein said formaldehyde is generated in situ via chemical reaction of one or more formaldehyde precursors.

38. The process of claim 37 wherein said one or more formaldehyde precursors is methanol or paraformaldehyde.

39. The process of claim 1 wherein said process is carried out in a reactor; wherein said formaldehyde is provided to said reactor as a mixture with methanol.

40. The process of claim 1 wherein said step of contacting formaldehyde with said N-heterocyclic carbene catalyst, providing said transition metal hydrogen-transfer catalyst in contact with at least a portion of said one or more reaction products or both steps of contacting formaldehyde with said N-heterocyclic carbene catalyst and providing said transition metal hydrogen-transfer catalyst in contact with at least a portion of said one or more reaction products is carried out in the presence of a base.

41. The process of claim 1 wherein said process is carried out in a reactor, said process further comprising the step of add one or more bases to said reactor.

42. A process for the production of ethylene glycol, said process comprising the steps of:
contacting formaldehyde with Enders carbene or a methanol adduct thereof, thereby generating one or more reaction products; and
providing Shvo's catalyst in contact with at least a portion of said one or more reaction products in the presence of methanol, thereby producing said ethylene glycol.

43. The process of claim 42, wherein said steps of contacting formaldehyde with Enders carbene or said methanol adduct thereof and providing Shvo's catalyst in contact with at least a portion of said one or more reaction products in the presence of methanol are carried out in a tetrahydrofuran solvent or dioxane solvent.
